SEQUENCE LISTING (12 sequences)

SEQ ID NO: 1 - protein, 108 aa, Homo sapiens
EEWFFKDITRKDAERQLLAPGNSAGAFLIRESETLKGSFSLSVRDFDPVHGDVIKHYKIRSLDNGGYYISPRITFPCISDMIKHYQKQADGLCRRLEKACISPKPQKP

SEQ ID NO: 2 - protein, 108 aa, Homo sapiens
EEWFFKDITRKDAERQLLAPGNSAGAFLIRESETLKGSFALSVRDFDPVHGDVIKHYLIRSLDNGGYYISPRITFPCISDMIKHYQKQADGLCRRLEKACISPKPQKP

SEQ ID NO: 3 - protein, 108 aa, Mus musculus
EEWFFKDITRKDAERQLLAPGNSAGAFLIRESETLKGSFSLSVRDYDPMHGDVIKHYKIRSLDNGGYYISPRITFPCISDMIKHYQKQSDGLCRRLEKACISPKPQKP

SEQ ID NO: 4 - protein, 108 aa, Rattus norvegicus; identical to SEQ ID NO: 3

SEQ ID NO: 5 - protein, 108 aa, Gallus gallus
EEWFFKDITRKDAERQLLAPGNGPGAFLIRESETLKGSYSLSIRDYDPQHGDVIKHYKIRSLDNGGFYISPRITFPSINDMIKHYQKQSDGLCRKLEKACISPKPQKP

SEQ ID NO: 6 - protein, 108 aa, Sus scrofa
EEWFFKDITRKDAERQLLAPGNSPGAFLIRESETLKGSYSLSVRDYDPVHGDVIKHYKIRSLDNGGYYISPRITFPCISDMIKYYQKQSDGLCRRLEKACISPKPQKP

SEQ ID NO: 7 - protein, 108 aa, Homo sapiens
WYAGACDRKSAEEALHRSNKDGSFLIRKSSGHDSKQPYTLVVFFNKRVYNIPVRFIEATKQYALGRKKNGEEYFGSVAEIIRNHQHSPLVLIDSQNNTKDSTRLKYAV

SEQ ID NO: 8 - protein, 108 aa, Homo sapiens
WYAGACDRKSAEEALHRSNKDGSFLIRKSSGHDSKQPYALVVFFNKRVYNIPVRFIEATKQYALGRKKNGEEYFGSVAEIIRNHQHSPLVLIDSQNNTKDSTRLKYAV

SEQ ID NO: 9 - protein, 111 aa, Mus musculus
WYAGACDRKSAEEALHRSNKDGSFLIRKSSGHDSKQPYTLVAFFNKRVYNIPVRFIEATKQYALGKKKNGEEYFGSVVEIVNSHQHNPLVLIDSQNNTKDSTRLKYAVKVS

SEQ ID NO: 10 - protein, 111 aa, Rattus norvegicus
WYAGACDRKSAEEALHRSNKDGSFLIRKSSGHDSKQPYTLVAFFNKRVYNIPVRFIEATKQYALGKKKNGEEYFGSVVEIIKNHQHNPLVLIDSQNNTKDSTRLKYAVKVS

SEQ ID NO: 11 - protein, 109 aa, Gallus gallus
WYAATCDRKTAEDALYRSNKDGSFLIRKSSGQDSRQPYTLVVFYNRRVYNIPIRFIESTRQYALGREKCGEERFDSVAEIVENHQHTSLVLIDSQNNTKDSTKLKYIVR

SEQ ID NO: 12 - protein, 111 aa, Sus scrofa
WYAGACDRKSAEEALYKSNKDGSFLIRKSSGHDSKQPYTLVVFFNKRVYNIPVRFIEATKQYALGRKKNGEEYFGSVAEIIKNHQHSPLVLIDSQNNTKDSTRLKYAVKVS

(19) United States
(12) Patent Application Publication
(10) Pub. No.: US 2021/0080465 A1
(43) Pub. Date: Mar. 18, 2021

(54) SH2 domain-based prognostic biomarker for chronic lymphocytic leukemia

(71) Applicant: UNIVERSITY OF CONNECTICUT, Farmington, CT (US)

(72) Inventors: Bruce Mayer, Farmington, CT (US); Kazuya Machida, Farmington, CT (US); Adam Lafontaine, Farmington, CT (US)

(21) Appl. No.: 17/023,652

(22) Filed: Sep. 17, 2020

Related U.S. Application Data
(60) Provisional application No. 62/901,901, filed on Sep. 18, 2019.

(51) Int. Cl. G01N 33/574

(52) U.S. Cl. CPC G01N 33/57426

(57) ABSTRACT

The present disclosure provides methods and compositions for prognosing Chronic Lymphocytic Leukemia (CLL) or monitoring CLL therapy in a subject, by contacting a blood sample from a subject having CLL with one or more tyrosine phosphorylation (pTyr) probes to promote binding of tyrosine phosphorylated proteins in the blood sample to the one or more pTyr probes to generate pTyr probe-protein complexes; and detecting an amount of pTyr probe-protein complexes in the blood sample; wherein an amount of pTyr probe-protein complexes in the blood sample is compared to a control to indicate expected progression of CLL, or therapeutic efficacy of CLL therapy in the subject.

### CROSS REFERENCE

This application claims priority to U.S. Provisional Patent Application Ser. No. 62/901901 filed Sep. 18, 2019, incorporated by reference herein in its entirety.

### STATEMENT OF GOVERNMENT RIGHTS

This invention was made with government support under grant numbers CA154966, CA146843 and CA107785 awarded by the National Institutes of Health. The government has certain rights in the invention.

### STATEMENT REGARDING SEQUENCE LISTING

This application contains a Sequence Listing submitted as an electronic text file named “19-1639-US_SequenceListing_ST25.txt”, having a size in bytes of 13 kb, and created on Sep. 11, 2020. The information contained in this electronic file is hereby incorporated by reference in its entirety pursuant to 37 CFR § 1.52(e)(5).

### BACKGROUND

Tyrosine phosphorylation controls many normal cell behaviors including proliferation, differentiation, survival, and motility, and deregulation of these pathways is a frequent driver of tumorigenesis. Changes in tyrosine phosphorylation impact downstream signaling primarily through changes in protein-protein interactions, which are mediated by phosphotyrosine (pTyr)-dependent modular binding domains such as SH2 domains.

### SUMMARY

In one aspect, the disclosure provides methods for prognosing Chronic Lymphocytic Leukemia (CLL) in a subject, comprising

(a) contacting a blood sample from a subject having CLL with one or more tyrosine phosphorylation (pTyr) probes under conditions to promote binding of tyrosine phosphorylated proteins in the blood sample to the one or more pTyr probes to generate pTyr probe-protein complexes; and

(b) detecting an amount of pTyr probe-protein complexes in the blood sample;

wherein an amount of pTyr probe-protein complexes in the blood sample is compared to a control to indicate expected progression of CLL in the subject.

In another aspect, the disclosure provides methods for monitoring therapy in a subject being treated for CLL, comprising

(a) contacting a blood sample from a subject receiving therapy for CLL with one or more pTyr probes under conditions to promote binding of tyrosine phosphorylated proteins in the blood sample to the one or more pTyr probes to generate pTyr probe-protein complexes; and

(b) detecting an amount of pTyr probe-protein complexes in the blood sample;

wherein an amount of pTyr probe-protein complexes in the blood sample is compared to a control to indicate efficacy of the therapy in the subject.

In one embodiment, the amount of pTyr probe-protein complexes in the blood sample is compared to a control to indicate that the subject has acquired resistance to the therapy. In another embodiment, the one or more pTyr probes comprise one or more Src Homology 2 (SH2) domain. In a further embodiment, the one or more pTyr probes comprise (i) one or more BLNK SH2 domains, and/or (ii) one or more LYN SH2 domains. In one embodiment, the one or more pTyr probes comprise (i) one or more BLNK SH2 domains, and/or (ii) one or more LYN SH2 domains, comprising or consisting of an amino acid sequence at least 80%, 85%, 90%, 91%, 92%, 93%, 94%, 95%, 96%, 97%, 98%, 99%, or 100% identical to the amino acid sequence of a BLNK domain selected from the group consisting of SEQ ID NOS:7-12 or a LYN SH2 domain selected from the group consisting of SEQ ID NOS:1-6, or combinations thereof.

In one embodiment, a combination of BLNK SH2-protein complexes below control levels and LYN SH2-protein complexes above control levels indicates that the subject is at high risk for CLL progression, or indicates that the therapy is ineffective. In another embodiment, a combination of BLNK SH2-protein complexes below control levels and LYN SH2-protein complexes below control levels indicates that the subject is at intermediate risk for CLL progression, or indicates that the therapy is partially ineffective. In a further embodiment, a level of BLNK SH2-protein complexes above control levels indicates that the subject is at low risk for CLL progression, or indicates that the therapy is effective. In one embodiment, the method further comprises modifying treatment of the subject based on the expected progression or efficacy of therapy indicated by the method.

In another aspect the disclosure provides compositions comprising:

(i) one or more BLNK SH2 domains, and

(ii) one or more LYN SH2 domains.

In one embodiment, the one or more BLNK SH2 domains comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NOS: 7-12, and the one or more LYN SH2 domains comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NOS:1-6. In another embodiment, the composition in total includes no more than 1000, 500, 250, 100, 75, 50, 25, 15, 14, 13, 12, 11, 10, 9, 8, 7, 6, 5, 4, 3, or 2 polypeptides. In a further embodiment, the one or more BLNK SH2 domains and the one or more LYN SH2 domains are detectably labeled. In one embodiment, the one or more BLNK SH2 domains and the one or more LYN SH2 domains are attached to a solid support.

### DESCRIPTION OF THE FIGURES

FIG. 1(A-C). SH2 binding patterns correlate with clinical outcomes. (A) SH2 profiling was performed on 35 CLL samples (first sample, taken at diagnosis) and quantitative SH2 binding data were subjected to hierarchical clustering. Heat map indicates relative binding. Current clinical status of each is indicated. PFS=months of progression free survival. IGHV=immunoglobulin heavy chain hypermutation, current standard-of-care prognostic marker for CLL (“mutated”, >than 2% relative to germline sequence). Black arrow: sample for which IGHV status gave false negative result. White arrow: long-term progression-free survival in sample predicted to have poor prognosis by IGHV status (apparent false positive). In both cases BLNK SH2 binding correctly predicted clinical outcome. (B) Disease course during clinical follow-up describing progression-free status of study subjects. (C) Obvious change of the SH2 profile for a CLL case was observed following treatment. This patient has now had a recurrence as predicted by SH2 profile.

FIG. 2. BLNK and LYN SH2 binding predicts clinical outcomes in CLL. Kaplan-Meier plots of the probability of progression-free survival for patients classified by BLNK and LYN SH2 binding status. BLNK low, LYN high is correlated with poor outcome (relatively short progression-free survival). BLNK low, LYN low is correlated with progression at later time points (intermediate prognosis). BLNK high predicts good prognosis, independent of LYN status. Time is indicated in months.

FIG. 3A-C. BLNK SH2 binding correlates with progression-free survival. Random Forest machine learning algorithm was used to assess correlation of SH2 binding data with clinical outcomes (progression-free survival vs. either progression or death). At all time points tested (A) 20 months, (B) 60 months, and (C) 100 months follow-up, low BLNK SH2 binding (upper left graph in each panel) was highly correlated with poor survival, i.e. low likelihood of progression-free survival at that time point.

FIG. 4A-B. Comparison of (A) IGVH mutation and (B) SH2 profiling status for predicting clinical outcomes in CLL. Kaplan-Meier plots of the probability of progression-free survival are shown for BLNK status (BLNK low, <0.3; BLNK high, >0.3) on left, and immunoglobulin heavy chain hyper-mutation status (umutated, <2% mutation relative to germ-line; mutated, either >2% mutation relative to germline or polyclonal). SH2 binding status correlates more highly with progression-free survival than IGHV status. Time is indicated in months. Shaded areas=95% confidence interval.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M.P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

The inventors have demonstrated that SH2 binding profiles correlate with clinical outcomes such as risk of progression prognosis and response to therapy in subjects with chronic lymphocytic leukemia (CLL). CLL is characterized by a relatively long asymptomatic chronic phase, followed with variable time-course by progression to acute disease, for which several targeted therapies are now available. Thus when to treat and what agents will be most effective are key clinical questions, and identifying predictive biomarkers could significantly impact outcomes for these patients.

In one aspect, the disclosure provides methods for prognosing Chronic Lymphocytic Leukemia (CLL) in a subject, comprising

(a) contacting a blood sample from a subject having CLL with one or more tyrosine phosphorylation (pTyr) probes under conditions to promote binding of tyrosine phosphorylated proteins in the blood sample to the one or more pTyr probes to generate pTyr probe-protein complexes; and

(b) detecting an amount of pTyr probe-protein complexes in the blood sample; wherein an amount of pTyr probe-protein complexes in the blood sample is compared to a control to indicate expected progression of CLL in the subject.

As used herein, “prognosing” includes predicting progression of CLL in the subject, predicting subject response to therapy including but not limited BCR (B-cell receptor) signaling inhibitors, etc., detecting disease progression, and/or identifying high or intermediate risk groups at diagnosis.

In another aspect, the disclosure provides methods for monitoring therapy in a subject being treated for CLL, comprising

(a) contacting a blood sample from a subject receiving therapy for CLL with one or more pTyr probes under conditions to promote binding of tyrosine phosphorylated proteins in the blood sample to the one or more pTyr probes to generate pTyr probe-protein complexes; and

(b) detecting an amount of pTyr probe-protein complexes in the blood sample;

wherein an amount of pTyr probe-protein complexes in the blood sample is compared to a control to indicate efficacy of the therapy in the subject.

As used herein, “monitoring therapy” includes monitoring effect on the subject of the treatment (i.e.: improvement, no change, decline, acquiring resistance to therapy, complete remission, partial remission, stable disease, progressive disease, etc.) by monitoring changing patterns of pTyr probe-protein complexes, reduction or disappearance of pTyr probe-protein complex patterns indicative of a high risk subject, etc.

In one embodiment, the amount of pTyr probe-protein complexes in the blood sample is compared to a control to indicate that the subject has acquired resistance to the therapy. Any suitable blood sample may be used, including but not limited to protein extracts from whole blood or cells within such blood samples, including but not limited to peripheral blood mononuclear cells (PBMCs).

Contacting of the blood sample may be done via any suitable procedure as deemed appropriate. In some embodiments, the contacting may be done in solution or proteins from a blood sample protein extract may be immobilized to a solid support, including but not limited to membranes (e.g. PVDF or nitrocellulose), plastic surfaces (e.g. polystyrene) or can be covalently coupled to appropriate beads (e.g. Epoxy-activated beads). For example, protein samples can be directly spotted on a membrane (dot/slot blot) or separated by conventional SDS-PAGE prior to the transfer on a membrane. Separation of proteins prior to analysis will provide qualitative in addition to quantitative information that will be helpful for the characterization of protein-protein interaction profiles and identification of specific binding partners especially when complex protein mixtures like clinical samples are investigated. The proteins from the blood sample may be denatured or non-denatured. In another embodiment, the contacting may comprise binding to intact fixed cells. This could comprise, for example, cells affixed to slides, or flow cytometry.

In all aspects and embodiments disclosed herein, the pTyr probes may be any suitable such probe that selectively binds to tyrosine-phosphorylated proteins (p-Tyr proteins), including but not limited to Src Homology 2 (SH2) domains and Phosphotyrosine binding (PTB) domains, or other modular p-Tyr binding domains

In a specific embodiment, the pTyr probes comprise or consist of Src Homology 2 (SH2) domains. SH2 profiling uses the cell pTyr signal response apparatus to interrogate the state of pTyr signaling. Upon receptor tyrosine kinase (RTK) activation, the resulting increase in protein tyrosine phosphorylation generates binding sites for modular pTyr-specific binding domains; it is the relocalization of intracellular effectors containing pTyr binding domains to these phosphorylated sites that is the key step in signal transmission. The most abundant pTyr binding module in humans is the Src Homology 2 or SH2 domain. There are at least 120 SH2 domains encoded by the human genome, and each SH2 domain binds a unique spectrum of tyrosine phosphorylated sites. Because SH2 domains are what the cell uses to respond to or “read” changes in tyrosine phosphorylation during signaling, the extent of binding of different SH2 domains can provide a wealth of information about the mechanisms and status of pTyr signaling.

In one embodiment, the one or more pTyr probes comprise (i) one or more BLNK SH2 domains, and/or (ii) one or more LYN SH2 domains. Any suitable BLNK SH2 domain and/or LYN SH2 domain may be used. Exemplary BLNK and LYN SH2 domains are provided herein. In specific embodiments, the one or more pTyr probes comprise (i) one or more BLNK SH2 domains, and/or (ii) one or more LYN SH2 domains comprising or consisting of an amino acid sequence at least 80%, 85%, 90%, 91%, 92%, 93%, 94%, 95%, 96%, 97%, 98%, 99%, or 100% identical to the amino acid sequence selected from the group consisting of BLNK (SEQ ID NOS:7-12) or LYN (SEQ ID NOS:1-6) SH2 domains.

Exemplary SH2 domains
Human LYN (NP_001104567.1):
(SEQ ID NO: 1)
EEWFFKDITRKDAERQLLAPGNSAGAFLIRESETLKGSFSLSVRDFDPVH
GDVIKHYKIRSLDNGGYYISPRITFPCISDMIKHYQKQADGLCRRLEKAC
ISPKPQKP
Human ultra-sensitive LYN (NP_001104567.1)
(SEQ ID NO: 2)
EEWFFKDITRKDAERQLLAPGNSAGAFLIRESETLKGSFALSVRDFDPVH
GDVIKHYLIRSLDNGGYYISPRITFPCISDMIKHYQKQADGLCRRLEKAC
ISPKPQKP
Mouse LYN (NP_001104566.1)
(SEQ ID NO: 3)
EEWFFKDITRKDAERQLLAPGNSAGAFLIRESETLKGSFSLSVRDYDPMH
GDVIKHYKIRSLDNGGYYISPRITFPCISDMIKHYQKQSDGLCRRLEKAC
ISPKPQKP
Rat LYN (NP_001104568.1)
(SEQ ID NO: 4)
EEWFFKDITRKDAERQLLAPGNSAGAFLIRESETLKGSFSLSVRDYDPMH
GDVIKHYKIRSLDNGGYYISPRITFPCISDMIKHYQKQSDGLCRRLEKAC
ISPKPQKP
Chicken LYN (NP_001006390.1)
(SEQ ID NO: 5)
EEWFFKDITRKDAERQLLAPGNGPGAFLIRESETLKGSYSLSIRDYDPQH
GDVIKHYKIRSLDNGGFYISPRITFPSINDMIKHYQKQSDGLCRKLEKAC
ISPKPQKP
Pig LYN (XP_020945022.1)
(SEQ ID NO: 6)
EEWFFKDITRKDAERQLLAPGNSPGAFLIRESETLKGSYSLSVRDYDPVH
GDVIKHYKIRSLDNGGYYISPRITFPCISDMIKYYQKQSDGLCRRLEKAC
ISPKPQKP
Human BLNK (NP_001107566.1)
(SEQ ID NO: 7)
WYAGACDRKSAEEALHRSNKDGSFLIRKSSGHDSKQPYTLVVFFNKRVYN
IPVRFIEATKQYALGRKKNGEEYFGSVAEIIRNHQHSPLVLIDSQNNTKD
STRLKYAV
Human ultra-sensitive BLNK (NP_001107566.1)
(SEQ ID NO: 8)
WYAGACDRKSAEEALHRSNKDGSFLIRKSSGHDSKQPYALVVFFNKRVYN
IPVRFIEATKQYALGRKKNGEEYFGSVAEIIRNHQHSPLVLIDSQNNTKD
STRLKYAV
Mouse BLNK (NP_001351983.1)
(SEQ ID NO: 9)
WYAGACDRKSAEEALHRSNKDGSFLIRKSSGHDSKQPYTLVAFFNKRVYN
IPVRFIEATKQYALGKKKNGEEYFGSVVEIVNSHQHNPLVLIDSQNNTKD
STRLKYAVKVS
Rat BLNK (NP_001020938.1)
(SEQ ID NO: 10)
WYAGACDRKSAEEALHRSNKDGSFLIRKSSGHDSKQPYTLVAFFNKRVYN
IPVRFIEATKQYALGKKKNGEEYFGSVVEIIKNHQHNPLVLIDSQNNTKD
STRLKYAVKVS
Chicken BLNK (XP_015144057.1)
(SEQ ID NO: 11)
WYAATCDRKTAEDALYRSNKDGSFLIRKSSGQDSRQPYTLVVFYNRRVYN
IPIRFIESTRQYALGREKCGEERFDSVAEIVENHQHTSLVLIDSQNNTKD
STKLKYIVR
Pig BLNK (XP_001928268.1)
(SEQ ID NO: 12)
WYAGACDRKSAEEALYKSNKDGSFLIRKSSGHDSKQPYTLVVFFNKRVYN
IPVRFIEATKQYALGRKKNGEEYFGSVAEIIKNHQHSPLVLIDSQNNTKD
STRLKYAVKVS

Any control may be used as suitable for a given use of the methods of the disclosure. In various embodiments, the control may be a blood sample from a subject not having CLL (such as a subject not having cancer), a base line blood sample from a CLL subject prior to CLL treatment initiation, a threshold value based on blood samples from a population of subjects not having CLL (such as a subject not having cancer) or blood samples from a population of CLL subjects prior to CLL treatment, stimulated or unstimulated B-cell line samples, internal phosphopeptide standards, etc.

As shown in the examples (“low” or “high” designations mean above or below a control level, including but not limited to a threshold level):

(1) The combination of Low BLNK SH2-protein complexes +High LYN SH2-protein complexes is correlated with poor outcome (relatively short progression-free survival).

(2) The combination of Low BLNK SH2-protein complexes +Low LYN SH2-protein complexes is correlated with progression at later time points (intermediate prognosis).

(3) High BLNK SH2-protein complexes predicts good prognosis, independent of LYN status.

Thus, in one embodiment, a combination of BLNK SH2-protein complexes below control levels and LYN SH2-protein complexes above control levels indicates that the subject is at high risk for CLL progression, or indicates that the therapy is ineffective.

In another embodiment, a combination of BLNK SH2-protein complexes below control levels and LYN SH2-protein complexes below control levels indicates that the subject is at intermediate risk for CLL progression, or indicates that the therapy is partially ineffective.

In a further embodiment, a level of BLNK SH2-protein complexes above control levels indicates that the subject is at low risk for CLL progression, or indicates that the therapy is effective.

The subject may be any suitable subject having CLL. In one embodiment, the subject is a human subject having CLL.

In another embodiment of any aspect herein, the methods further comprise modifying treatment of the subject based on the expected progression or efficacy of therapy indicated by the method. For example, in embodiments here (a) a combination of BLNK SH2-protein complexes below control levels and LYN SH2-protein complexes above control levels indicates that the subject is at high risk for rapid CLL progression, or indicates that the therapy is ineffective, or (b) a combination of BLNK SH2-protein complexes below control levels and LYN SH2-protein complexes below control levels indicates that the subject is at intermediate risk for CLL progression, or indicates that the therapy is partially ineffective, the methods may further comprise modifying treatment of the subject. The treatment modification may be any deemed suitable by attending medical personnel. In one embodiment, the modification comprises increasing dosage and/or number of administrations of the therapy being administered to the subject. In another embodiment, the modification comprises changing the therapy being administered to the subject (i.e.: substituting one therapy for another; adding a therapy to the existing therapy; etc.) In various non-limiting embodiments, the modification may comprise increasing the dose and/or number of administrations of a therapy, substituting the therapy being received by the subject, or combining the therapy being received by the subject, based on therapeutics including but not limited to nucleoside analogues including but not limited to fludarabine, cladribine, pentostatin, chlorambucil, or combinations thereof; combinations of monoclonal antibodies including but not limited to alemtuzumab, rituximab, ofatumumab, obinutuzumab, epratuzumab, and lumiliximab or combinations thereof; other therapeutics including but not limited to duvelisib, ibrutinib, idelalisib, venetoclax, lenalidomide; allogeneic stem cell transplantation; and combination therapies including but not limited to:
Fludarabine, cyclophosphamide, and rituximab (FCR)
Pentostatin, cyclophosphamide, and rituximab (PCR)
Fludarabine, cyclophosphamide, and mitoxantrone (FCM)
Cyclophosphamide, vincristine, and prednisone (CVP)
Cyclophosphamide, doxorubicin, vincristine, and prednisone (CHOP)
Cladribine and/or fludarabine, in combination with cyclophosphamide
Bendamustine and rituximab combination;
Fludarabine and rituximab
FCR and alemtuzumab;
Ofatumumab and chlorambucil;
Ofatumumab, fludarabine, and cyclophosphamide;
Obinutuzumab and chlorambucil;
Alemtuzumab and fludarabine.

The assays and detecting steps may be carried out using any suitable methods, including but not limited to those disclosed in U.S. Pat. No. 7,846,746, incorporated by reference herein it its entirety. For example:

For the detection and characterization of specific protein-protein interactions, the pTyr probe may be labeled to facilitate detection. Depending on the labeling strategy, a competitive binding assay may be performed in two exemplary ways. In one embodiment, only one pTyr probe is labeled while all other pTyr probes in the reaction mixture (if any) remain unlabeled. Under these conditions, several independent binding reactions can be performed with the same immobilized protein to compare the patterns with different binding domains. The number of separate binding experiments depends on the number of the pTyr probes that are applied for the assay. The different binding reactions can be performed either in parallel (for example, with multiple filters on which the same type and amount of protein is immobilized) or by repeated cycles of binding (for example, with, the same filter after the removal of the labeled pTyr probe which was applied in the previous binding reaction). In a second embodiment, multiple SH2 domains are differentially labeled (for example, with different fluorescent probes) and binding of multiple SH2 domains is detected simultaneously in the same sample.

Labeling of pTyr probes and their detection can be achieved in several ways. For example, a tag may be fused to a pTyr probe and used for detection (e.g. glutathione-S-transferase (GST) tag, biotinylation, antibody recognition sequences, etc.). Alternatively, pTyr probes can be labeled after purification e.g. by biotinylation or by the covalent attachment of fluorophores.

In another aspect, the disclosure provides compositions comprising:

(i) one or more BLNK SH2 domains, and

(ii) one or more LYN SH2 domains.

The compositions may be used in the methods of the disclosure. Any suitable BLNK SH2 domain and/or LYN SH2 domain may be used. Exemplary BLNK and LYN SH2 domains are provided herein. In one embodiment, the one or more BLNK SH2 domains comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NOS:7-12, and the one or more LYN SH2 domains comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NOS:1-6. In one embodiment, the composition in total includes no more than 1000, 500, 250, 100, 75, 50, 25, 15, 14, 13, 12, 11, 10, 9, 8, 7, 6, 5, 4, 3, or 2 polypeptides.

In one embodiment, the one or more BLNK SH2 domains, and the one or more LYN SH2 domains may be detectably labeled. Any detectable label may be used as deemed appropriate for an intended use. In one embodiment, the one or more BLNK SH2 domains and the one or more LYN SH2 domains are differentially labeled (for example, with different fluorescent probes). Labeling of the domains and their detection can be achieved in several ways. For example, a tag may be fused to the domains and used for detection (e.g. glutathione-S-transferase (GST) tag, biotinylation, antibody recognition sequences, etc.). Alternatively, the domains can be labeled by biotinylation or by the covalent attachment of fluorophores. In all embodiments, the BLNK and LYN domains may be provided in any format, including but not limited to in solution or attached to a solid support. Such solid supports may include, but are not limited to, membranes (e.g. PVDF or nitrocellulose), plastic surfaces (e.g. polystyrene), beads (e.g. Epoxy-activated beads), microarrays, microplates, etc.

### EXAMPLES

We describe an assay based on quantitative binding of a Src Homology 2 (SH2) domain probe to proteins in blood samples from patients with chronic lymphocytic leukemia (CLL) that predicts progression-free survival for those patients. This information is clinically useful in stratifying patients and identifying those who should be treated aggressively. This and related biomarkers are likely to also be useful in predicting response to specific targeted therapies, monitoring response to therapy, and monitoring acquired resistance to therapy. Biomarkers based on tyrosine phosphorylation state (SH2 profiling) are likely to be more accurate than current tests because they assess the global state of signaling that drives the tumor, as opposed to monitoring a single gene, protein, or mutation as in current markers. The described biomarkers could be the basis for clinical blood tests routinely used to guide the treatment of patients with CLL.

The biomarker is based on binding of SH2 domains to cellular tyrosine phosphorylated proteins. This interaction is key to signaling that drives the tumor. Other assays monitor nucleic acid mutations, gene overexpression, or activation of specific signaling proteins. The basis for the current assay is conceptually very different, and is complementary to and distinct from other approaches.

In most cases, tyrosine phosphorylation transmits signals by creating binding sites for pTyr binding domains such as Src Homology 2 (SH2) domains; this results in the recruitment of SH2-containing effector proteins and the assembly of membrane-associated signaling complexes. In humans, there are ˜120 SH2 domains, and a few other pTyr binding domains including several PTB domains. Each domain binds to a distinct but overlapping set of tyrosine phosphorylated peptide sites. Because the cell's ability to read and decode changes in tyrosine phosphorylation depends entirely on this set of pTyr binding domains, the quantitative levels of their binding sites provide an excellent global representation of the state of pTyr signaling.

Results: We have discovered SH2 binding domain patterns from patient blood samples predictive of clinical outcome.

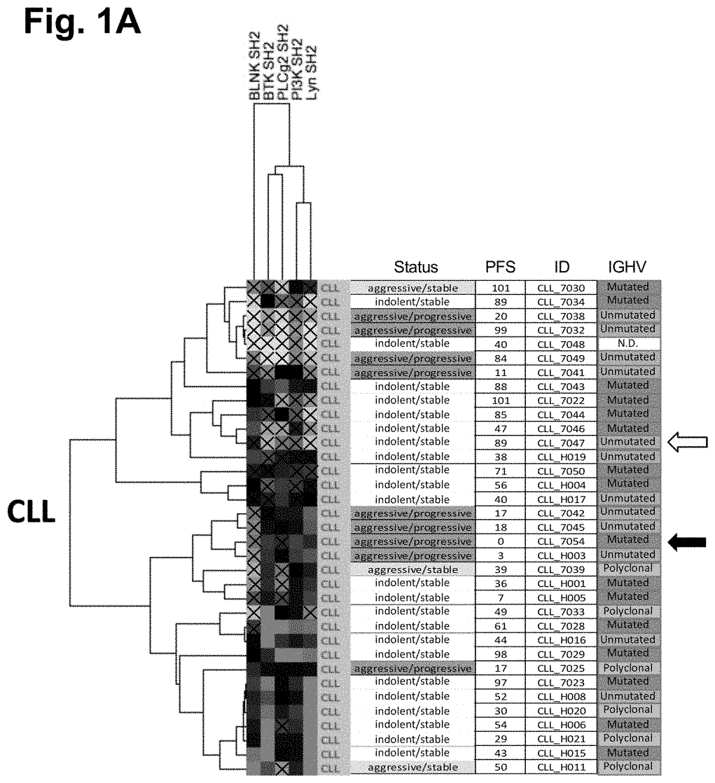

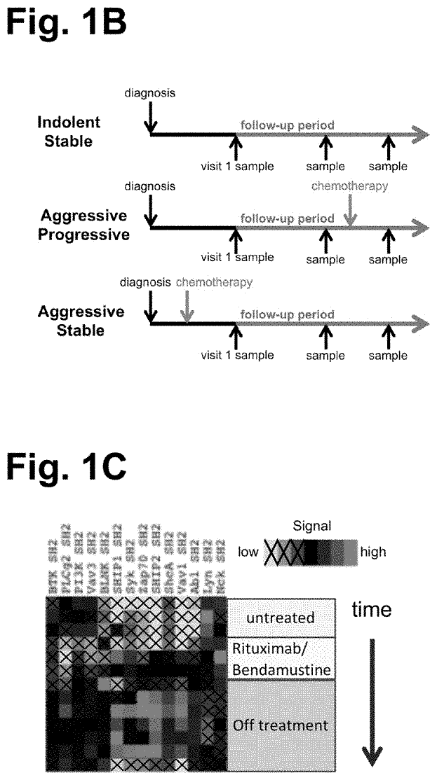

SH2 profiling of patient samples: We have been archiving CLL patient samples from Hartford area hospitals for over 10 years. Blood is collected from patients on initial diagnosis and at each subsequent follow-up visit (˜six month intervals). Peripheral blood mononuclear cells (PBMC) are prepared and cells divided into two fractions; one is lysed for protein analysis, and the second is frozen for cell culture. ˜350 samples from over 50 unique patients have been collected to date, and collection of new samples is ongoing. Sample preparation and assay methodologies have been optimized to preserve tyrosine phosphorylation for SH2 profiling (1-4). Clinical information is provided to investigators in a de-identified manner. The outline of clinical outcome is shown in FIG. 1B.

Study design: We performed statistical analysis on a subset of our patient data to see if specific patterns of SH2 binding data can be correlated to clinical outcome. Five SH2 domain probes specific to BCR signaling, namely BLNK, BTK, PI3K, PLCG2 and LYN, were used to screen 35 CLL samples (the first sample, provided at diagnosis, for each patient for whom subsequent samples were available). Samples were subjected to hierarchical clustering to assess their relatedness based on the quantification of SH2 binding sites. As can be seen in FIG. 1A, samples from patients with progressive disease grouped into two distinct clusters. One cluster (A), consisting of patients who progressed relatively rapidly, was characterized by low BLNK and high LYN SH2 binding; the second cluster (B), with longer time to progression, had low BLNK and low LYN binding. SH2 profiling was also used to follow response to therapy in the case of one patient (FIG. 1C). The SH2 profile changed dramatically upon therapy, and remained stable after therapy while the patient was in remission. Notably, the profile then reverted to the pre-treatment SH2 profile, and this patient has since suffered a relapse.

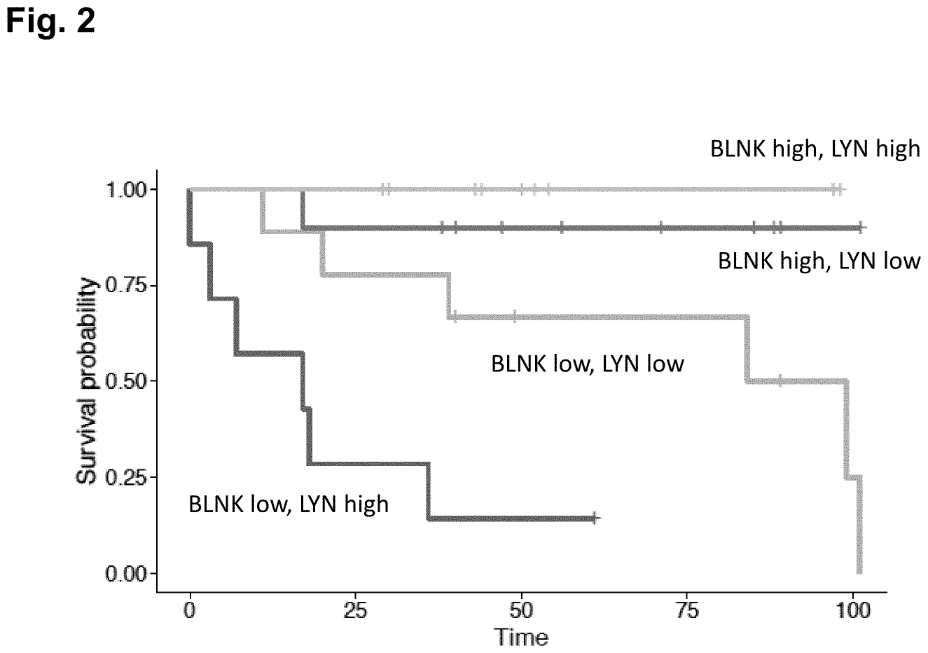

Based on the above findings, a Kaplan-Meier survival analysis was performed using the progression-free survival as the clinical endpoint (FIG. 2). Subjects were classified by BLNK and LYN SH2 binding status: group 1, LYN−BLNK− for low LYN and BLNK binding; group 2, LYN+BLNK—for high LYN and low BLNK binding; group 3, LYN−BLNK+for low LYN and high BLNK binding; group 4, LYN+BLNK+for high LYN and high BLNK binding. The group 2 (LYN+BLNK−) is correlated with poor outcome (relatively short progression-free survival). Group 1 (LYN−BLNK−) is correlated with progression at later time points (intermediate prognosis). BLNK high Groups 3 and 4 predict good prognosis, independent of LYN status. This result indicates classification of CLL patients based on SH2 pTyr probes predicts progression of the disease. BLNK, either alone or combination with LYN, shows a strong correlation with the progression-free survival.

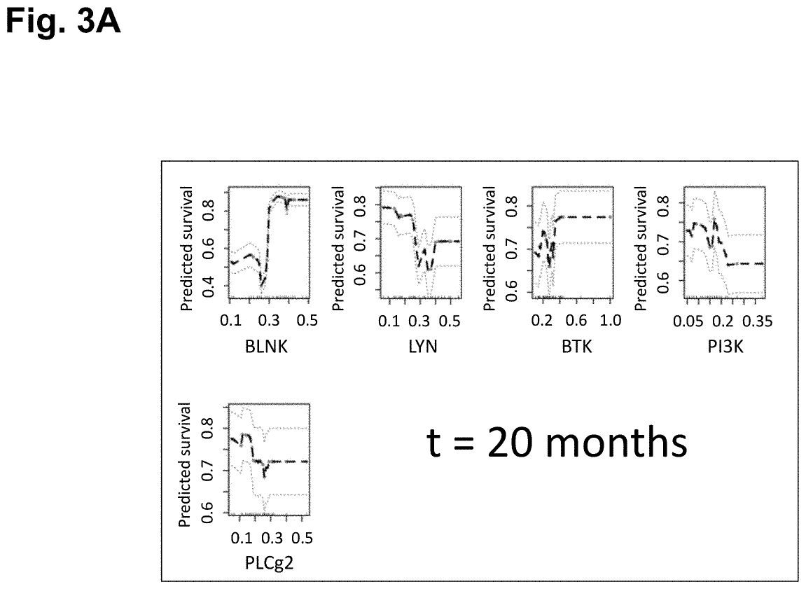

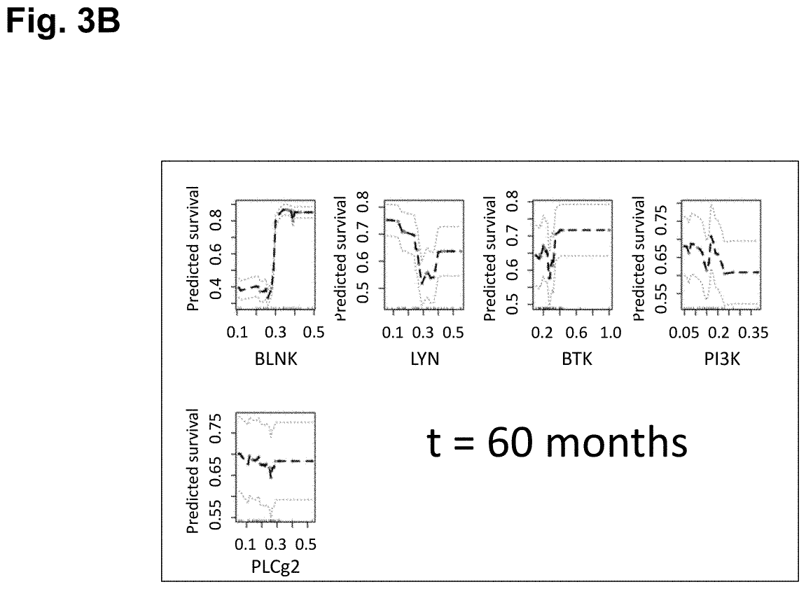

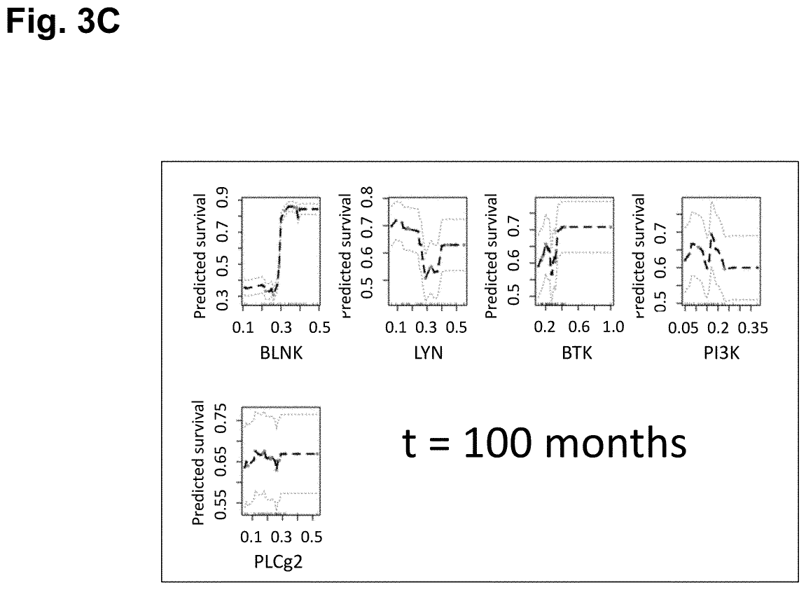

Next, we determined if variable thresholding of SH2 pTyr probe binding status can predict the subject outcome. Analysis using Random Forest (RF), a machine learning method of building classifiers, showed that low BLNK SH2 binding strongly correlated with low probability of progression-free survival (PFS) at 100 months (FIG. 3). Of these patients 13 (37%) have progressed or died, with a mean time of 35 months. Kaplan-Meier plots showed very strong correlation between BLNK SH2 binding sites and long-term survival (FIG. 2). Cox regression analysis showed BLNK binding to have a protective effect on PFS with a log hazard ratio of −6.5 associated with a one-unit change in binding (p value 0.02). With this dataset we can achieve 80% power, type-I error of 0.05 to detect a log hazards ratios of 7.2, 4.2, 10.5, 7.2, 5.2 associated with a one-unit change in BLNK, BTK, PI3K, PLCG2 and LYN respectively. Importantly, the predictive value of low BLNK SH2 binding was also seen at the 20 month and 60 month timepoints. This result confirms that BLNK SH2 alone has predictive value for the progression-free survival of CLL patients. We also compared Time-dependent Receiver Operating Characteristic (ROC) curves from censored survival data with the random forest model. The results suggest most of the predictive value for progression free survival is provided by the BLNK marker.(new figure).

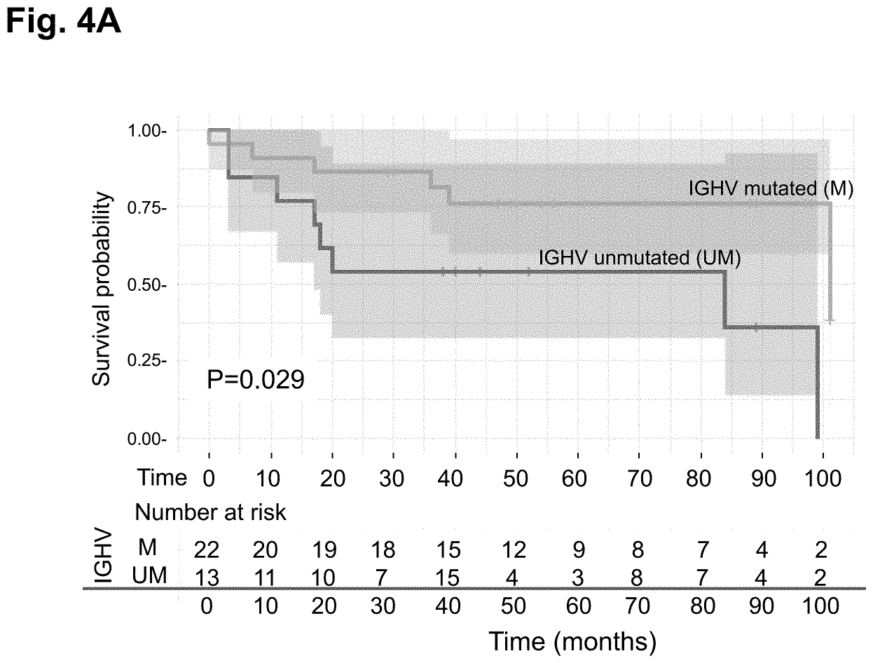

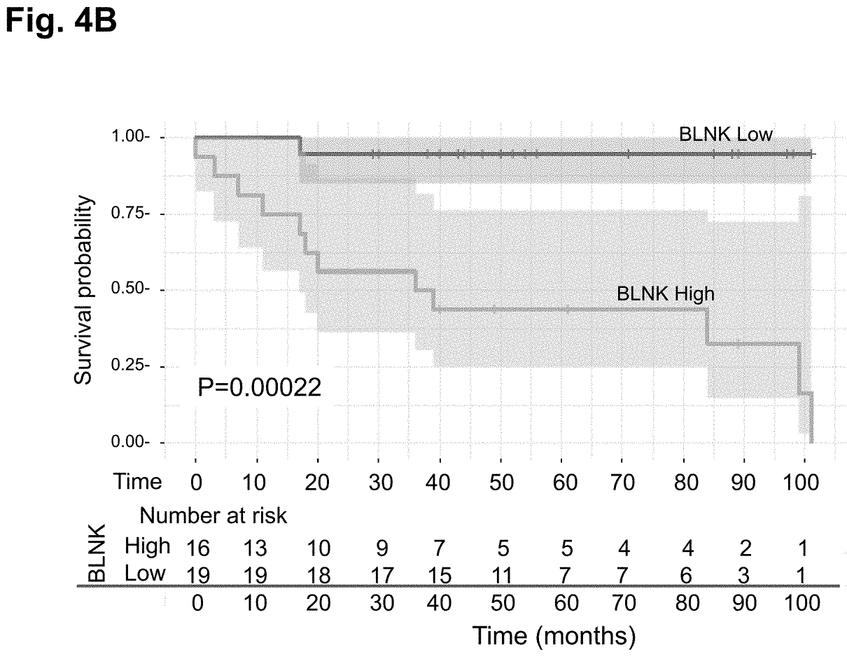

The immunoglobulin heavy chain hyper-mutation (IGHV) status of CLL cells is an established predictive biomarker which is known to be well correlated with patient survival; CLL patients with mutated IGHV show long-term disease-free survival, while unmated IGHV predicts early progression and poor survival (5). Hence we compared the SH2 profile (BLNK) and IGVH mutation status for predicting clinical outcomes in CLL. FIG. 4 shows Kaplan-Meier plots of the probability of progression-free survival for BLNK status (left panel, BLNK low, <0.3; BLNK high, >0.3) and IGHV (right panel, unmutated, <2% mutation relative to germ-line; mutated, either >2% mutation relative to germline or polyclonal). SH2 binding status correlates more highly with progression-free survival than IGHV status determined by 95% confidence intervals. Importantly, there are cases where the SH2 status was prognostic but the IGHV status did not accurately predict the high/low risk CLL subjects (FIG. 1A, arrows). Taken together these results indicate that BLNK and LYN SH2 domain binding patterns using patient blood samples predict clinical outcomes for CLL as well or better than the established IGHV biomarker.

### REFERENCES

1. Ng K Y, Machida K. Rosette Assay: Highly Customizable Dot-Blot for SH2 Domain Screening. Methods Mol Biol. 2017;1555:437-51. Epub 2017/01/17. doi: 10.1007/978-1-4939-6762-9 26. PubMed PMID: 28092049.
2. Machida K, Khenkhar M, Nollau P. Deciphering Phosphotyrosine-Dependent Signaling Networks in Cancer by SH2 Profiling. Genes & cancer. 2012;3(5-6):353-61. Epub 2012/12/12. doi: 10.1177/1947601912459048. PubMed PMID: 23226573.
3. Machida K, Eschrich S, Li J, Bai Y, Koomen J, Mayer B J, Haura E B. Characterizing tyrosine phosphorylation signaling in lung cancer using SH2 profiling. PLoS One.
2010;5(10):e13470. Epub 2010/10/27. doi: 10.1371/journal.pone.0013470. PubMed PMID: 20976048; PMCID: 2957407.
4. Machida K, Thompson C M, Dierck K, Jablonowski K, Karkkainen S, Liu B, Zhang H, Nash P D, Newman D K, Nollau P, Pawson T, Renkema G H, Saksela K, Schiller M R, Shin D G, Mayer B J. High-Throughput Phosphotyrosine Profiling Using SH2 Domains. Mol Cell. 2007;26(6):899-915. PubMed PMID: 17588523.
5. Hamblin T J, Davis Z, Gardiner A, Oscier D G, Stevenson F K. Unmutated Ig V(H) genes are associated with a more aggressive form of chronic lymphocytic leukemia. Blood. 1999;94(6):1848-54. Epub 1999/09/09. PubMed PMID: 10477713.
6. Fabbri G, Dalla-Favera R. The molecular pathogenesis of chronic lymphocytic leukaemia. Nature Reviews Cancer, 2016;16(3):145-162. PubMed PMID: 26911189.
7. Burger J A, Wiestner A. Targeting B cell receptor signalling in cancer: preclinical and clinical advances. Nature reviews Cancer, 2018;18(3):148-167. PubMed PMID: 29348577.

We claim:

1. A method for prognosing Chronic Lymphocytic Leukemia (CLL) in a subject, comprising
(a) contacting a blood sample from a subject having CLL with one or more tyrosine phosphorylation (pTyr) probes under conditions to promote binding of tyrosine phosphorylated proteins in the blood sample to the one or more pTyr probes to generate pTyr probe-protein complexes; and
(b) detecting an amount of pTyr probe-protein complexes in the blood sample;
wherein an amount of pTyr probe-protein complexes in the blood sample is compared to a control to indicate expected progression of CLL in the subject.

2. A method for monitoring therapy in a subject being treated for CLL, comprising
(a) contacting a blood sample from a subject receiving therapy for CLL with one or more pTyr probes under conditions to promote binding of tyrosine phosphorylated proteins in the blood sample to the one or more pTyr probes to generate pTyr probe-protein complexes; and
(b) detecting an amount of pTyr probe-protein complexes in the blood sample;
wherein an amount of pTyr probe-protein complexes in the blood sample is compared to a control to indicate efficacy of the therapy in the subject.

3. The method of claim 2, wherein the amount of pTyr probe-protein complexes in the blood sample is compared to a control to indicate that the subject has acquired resistance to the therapy.

4. The method of claim 1, wherein the one or more pTyr probes comprise one or more Src Homology 2 (SH2) domain.

5. The method of claim 1, wherein the one or more pTyr probes comprise (i) one or more BLNK SH2 domains, and/or (ii) one or more LYN SH2 domains.

6. The method of claim 1, wherein the one or more pTyr probes comprise (i) one or more BLNK SH2 domains, and/or (ii) one or more LYN SH2 domains, comprising or consisting of an amino acid sequence at least 80%, 85%, 90%, 91%, 92%, 93%, 94%, 95%, 96%, 97%, 98%, 99%, or 100% identical to the amino acid sequence of a BLNK domain selected from the group consisting of SEQ ID NOS:7-12 or a LYN SH2 domain selected from the group consisting of SEQ ID NOS:1-12, or combinations thereof.

7. The method of claim 5, wherein a combination of BLNK SH2-protein complexes below control levels and LYN SH2-protein complexes above control levels indicates that the subject is at high risk for CLL progression, or indicates that the therapy is ineffective.

8. The method of claim 5, wherein a combination of BLNK SH2-protein complexes below control levels and LYN SH2-protein complexes below control levels indicates that the subject is at intermediate risk for CLL progression, or indicates that the therapy is partially ineffective.

9. The method of claim 5, wherein a level of BLNK SH2-protein complexes above control levels indicates that the subject is at low risk for CLL progression, or indicates that the therapy is effective.

10. The method of claim 1, further comprising modifying treatment of the subject based on the expected progression or efficacy of therapy indicated by the method.

11. A composition comprising:
(i) one or more BLNK SH2 domains, and
(ii) one or more LYN SH2 domains.

12. The composition of claim 11, wherein the one or more BLNK SH2 domains comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NOS: 7-12, and the one or more LYN SH2 domains comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NOS:1-6.

13. The composition of claim 11, wherein the composition in total includes no more than 1000, 500, 250, 100, 75, 50, 25, 15, 14, 13, 12, 11, 10, 9, 8, 7, 6, 5, 4, 3, or 2 polypeptides.

14. The composition of claim 11, wherein the one or more BLNK SH2 domains and the one or more LYN SH2 domains are detectably labeled.

15. The composition of claim 11, wherein the one or more BLNK SH2 domains and the one or more LYN SH2 domains are attached to a solid support.
